(19) United States
(12) Patent Application Publication
(10) Pub. No.: US 2025/0281022 A1
(43) Pub. Date: Sep. 11, 2025

(54) ENDOSCOPY SUPPORT DEVICE, ENDOSCOPY SUPPORT METHOD, AND RECORDING MEDIUM

(71) Applicant: NEC Corporation, Tokyo (JP)

(72) Inventors: Takayuki OKUNO, Tokyo (JP); Naoto MAEDA, Tokyo (JP); Masahiro SAIKOU, Tokyo (JP)

(73) Assignee: NEC Corporation, Tokyo (JP)

(21) Appl. No.: 19/051,503

(22) Filed: Feb. 12, 2025

(30) Foreign Application Priority Data
Mar. 11, 2024 (JP) 2024-037057

(51) Int. Cl. A61B 1/00; G06T 7/00; G06T 7/11

(52) U.S. Cl. CPC A61B 1/00009; A61B 1/0002; A61B 1/0005; G06T 7/0012; G06T 7/11; G06T 2207/10004; G06T 2207/10068; G06T 2207/20081; G06T 2207/30096

(57) ABSTRACT

In an endoscopy support device, a display control means superimposes and displays a lesion guide for specifying a lesion in an endoscopic image. An acquisition means acquires the endoscopic image. An analysis means segments a lesion region from the endoscopic image based on the endoscopic image and the lesion guide. An output means outputs the lesion region segmented. The endoscopy support device can support decision making by a user in the field of healthcare.

### TECHNICAL FIELD

This disclosure relates to support for an endoscopic examination.

### BACKGROUND ART

An endoscopy support device is known to detect and identify lesions from image data taken during an endoscopic examination. For example, Patent document 1 proposes an inspection support device that can properly diagnose an area of interest to a doctor from the images taken by an endoscope.
Patent Document 1: Japanese Patent Application Laid-Open under No. 2022-145395

### SUMMARY

During an endoscopic examination, if a doctor finds a lesion, the doctor desires to identify a boundary area of a lesion in order to determine a depth of the lesion or to perform a biopsy.

One object of the present disclosure is to provide an endoscopy support device capable of identifying the boundary area of the lesion.

According to an example aspect of the present invention, there is provided an endoscopy support device including:
at least one memory configured to store instructions; and
at least one processor configured to execute the instructions to:
superimpose and display a lesion guide for specifying a lesion in an endoscopic image;
acquire the endoscopic image;
segment a lesion region from the endoscopic image based on the endoscopic image and the lesion guide; and
output the lesion region segmented.

According to another example aspect of the present invention, there is provided an endoscopy support method executed by a computer, including:
superimposing and displaying a lesion guide for specifying a lesion in an endoscopic image;
acquiring the endoscopic image;
segmenting a lesion region from the endoscopic image based on the endoscopic image and the lesion guide; and
outputting the lesion region segmented.

According to still another example aspect of the present invention, there is provided a non-transitory computer-readable recording medium storing a program causing a computer to execute processing of:
superimposing and displaying a lesion guide for specifying a lesion in an endoscopic image;
acquiring the endoscopic image;
segmenting a lesion region from the endoscopic image based on the endoscopic image and the lesion guide; and
outputting the lesion region segmented.

### EFFECT

According to this disclosure, it is possible to identify a boundary area of a lesion.

### BRIEF DESCRIPTION OF THE DRAWINGS

FIG. 1 is a block diagram illustrating a schematic configuration of an endoscopy system according to the present disclosure.

FIG. 2 is a block diagram illustrating a hardware configuration of an endoscopy support device according to the present disclosure.

FIG. 3 is a block diagram illustrating a functional configuration of the endoscopy support device according to the present disclosure.

FIG. 4 illustrates an example of a lesion guide.

FIG. 5 illustrates an example of a setting screen of the lesion guide.

FIG. 6 illustrates an example of a display of a boundary area of a lesion.

FIG. 7 is a flowchart of a process by the endoscopy support device according to the present disclosure.

FIG. 8 is a block diagram illustrating a functional configuration of another endoscopy support device according to the present disclosure.

FIG. 9A and FIG. 9B illustrate examples of a display of an identification result of the lesion.

FIG. 10 illustrates another example of the display of the identification result of the lesion.

FIG. 11 illustrates a further example of the display of the identification result of the lesion.

FIG. 12 is a block diagram illustrating a functional configuration of another endoscopy support device according to the present disclosure.

FIG. 13 is a flowchart of a process by the other endoscopy support device according to the present disclosure.

### EXAMPLE EMBODIMENTS

Preferred example embodiments of the present disclosure will be described with reference to the accompanying drawings.

### First Example Embodiment

### Overall Configuration

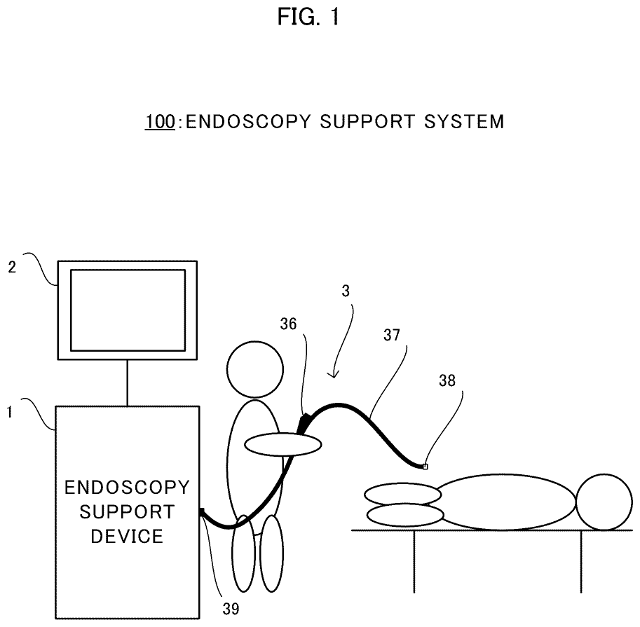

FIG. 1 shows a schematic configuration of an endoscopy system 100. The endoscopy system 100 superimposes a lesion guide on an endoscopic image being taken during an examination using an endoscope. The lesion guide is formed by frames and points. In a case where a doctor finds a lesion for which the doctor desires to identify a boundary area, the doctor manipulates the endoscope to fit the lesion within the frame of the lesion guide. Based on each endoscopic image and the lesion guide, the endoscopy system 100 segments a lesion region in the endoscopic image and displays the results. This allows the doctor to ascertain the boundary area of the lesion.

As shown in FIG. 1, the endoscopy system 100 mainly includes an endoscopy support device 1, a display device 2, and an endoscope 3 connected to the endoscopy support device 1.

The endoscopy support device 1 acquires, from the endoscope 3, each image (hereinafter, also referred to as an “endoscopic image Ic”) taken by the endoscope 3 during an endoscopic examination, and displays display data on the display device 2 for the endoscopic examiner (doctor) to check. Specifically, the endoscopy support device 1 acquires a video of an inside of an organ captured by the endoscope 3 as the endoscopic image Ic during the endoscopic examination. When the doctor finds a lesion during the endoscopic examination, the doctor manipulates the endoscope 3 and inputs a capturing instruction for capturing images of a location of the lesion. The endoscopy support device 1 captures and generates a lesion image of the lesion location based on the capturing instruction by the doctor. Specifically, the endoscopy support device 1 generates a still lesion image from each of endoscopic images Ic, which forms the video, based on the capturing instruction.

The display device 2 is a display or the like that performs a predetermined display based on a display signal supplied by the endoscopy support device 1.

The endoscope 3 mainly has a control unit 36 for the doctor to input an air supply, a water supply, an angle adjustment, a capturing instruction, etc., a flexible shaft 37 to be inserted into the organ to be examined by an examinee, a tip 38 with a built-in imaging unit such as an ultra-small imaging device, and a connection unit 39 for connecting to the endoscopy support device 1.

The following description is based on the assumption that a process is mainly used for colonoscopy. However, the examination target is not limited to a large intestine, but may also include a stomach, an esophagus, a small intestine, duodenum, and other digestive tracts (digestive organs).

### Hardware Configuration

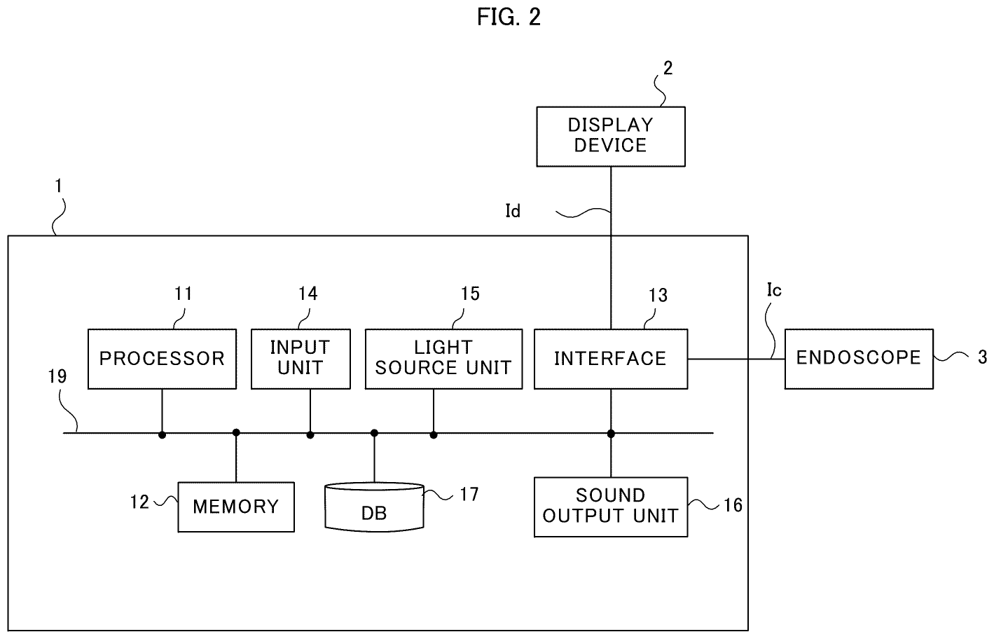

FIG. 2 shows a hardware configuration of the endoscopy support device 1. The endoscopy support device 1 mainly includes a processor 11, a memory 12, an interface 13, an input unit 14, a light source unit 15, a sound output unit 16, and a database (hereinafter, referred to as a “DB”) 17. Each of these elements is connected via a data bus 19.

The processor 11 executes a program, etc. stored in memory 12 to perform a predetermined process. The processor 11 is a processor such as a CPU (Central Processing Unit), a GPU (Graphics Processing Unit), a TPU (Tensor Processing Unit), etc. The processor 11 may be formed by a plurality of processors. The processor 11 is an example of a computer.

The memory 12 is formed by various volatile memories, such as a RAM (Random Access Memory) and a ROM (Read Only Memory), which are used as working memory, and one or more non-volatile memories which store information necessary for processing of the endoscopy support device 1. Note that the memory 12 may include an external storage device such as a hard disk connected to or built into the endoscopy support device 1, or may include a storage medium such as a removable flash memory or disk medium. The memory 12 stores programs for the endoscopy support device 1 to perform each process in this example embodiment.

Also, the memory 12 temporarily stores a series of endoscopic images Ic captured by the endoscope 3 in the endoscopic examination under a control of the processor 11. The memory 12 also temporarily stores each lesion image taken based on the capturing instruction by the doctor during the endoscopic examination. These images are stored in the memory 12 in association with, for example, identification information of the examinee (e.g., patient ID) and time stamp information.

The interface 13 performs interface operations between the endoscopy support device 1 and external devices. For example, the interface 13 supplies display data Id generated by the processor 11 on the display device 2. Moreover, the interface 13 supplies illumination light generated by the light source unit 15 to the endoscope 3. Furthermore, the interface 13 supplies an electrical signal indicating the endoscopic images Ic supplied from the endoscope 3 to the processor 11. The interface 13 may be a communication interface such as a network adapter for communicating with an external device by wire or wireless, or a hardware interface conforming to a USB (Universal Serial Bus), a SATA (Serial AT Attachment), etc.

The input unit 14 generates input signals based on the physician's operations. The input unit 14 is, for example, a button, a touch panel, a remote controller, a foot pedal, a voice input device, etc. The light source unit 15 generates light to be supplied to the tip 38 of the endoscope 3. Also, the light source unit 15 may incorporate a pump etc. to deliver water or air to supply the endoscope 3. The sound output unit 16 outputs sound based on the control of the processor 11.

The DB 17 stores a machine learning model such as a segmentation model described below. The DB 17 may include an external storage device such as a hard disk connected to or built into the endoscopy support device 1, or may include a storage medium such as a removable flash memory. Note that instead of having the DB 17 in the endoscopy system 100, the DB 17 may be provided on an external server etc., and data may be acquired from the external server etc. via communication.

### Functional Configuration

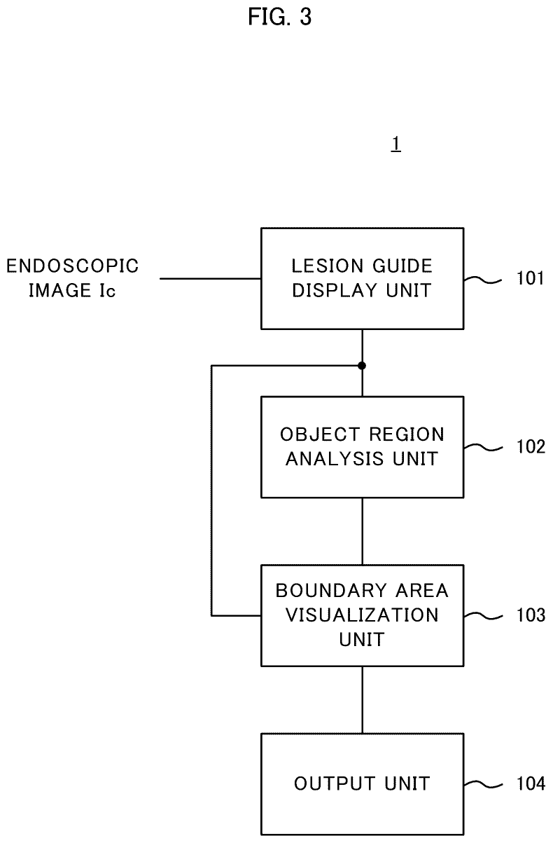

FIG. 3 is a block diagram showing a functional configuration of the endoscopy support device 1 of the first example embodiment. The endoscopy support device 1 functionally includes a lesion guide display unit 101, an object region analysis unit 102, a boundary area visualization unit 103, and an output unit 104.

Each endoscopic image Ic is input to the endoscopy support device 1 from the endoscope 3. Each endoscopic image Ic is input to the lesion guide display unit 101.

The lesion guide display unit 101 generates a lesion guide. The lesion guide serves as a landmark for the physician to fit the lesion within a predetermined area of the endoscopic image Ic. The lesion guide is formed by a frame or a point. The lesion guide display unit 101 generates display data using the endoscopic image Ic and the lesion guide, and outputs the display data to the display device 2.

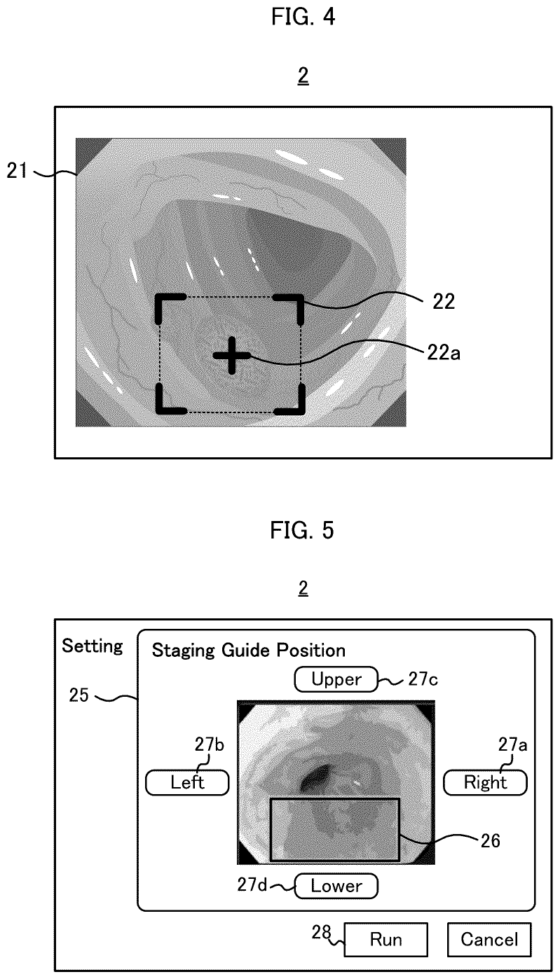

FIG. 4 shows an example of the lesion guide. As shown in FIG. 4, a current endoscopic image 21 is displayed on the display device 2, and a lesion guide 22 is superimposed on the endoscopic image 21. An interest point 22a indicates a center of the lesion guide 22.

Note that a display position of the lesion guide can be set arbitrarily. For example, the doctor can set a display position of a lesion guide 26 from a setting screen as illustrated in FIG. 5 before the endoscopic examination. The setting screen for the lesion guide in FIG. 5 includes a setting area 25 and an execution button 28. The doctor can move the lesion guide 26 up, down, left, or right by operating control buttons 27a, 27b, 27c, and 27d in the setting area 25. The doctor can then fix the display position of the lesion guide by pressing the execution button 28. This allows the doctor to set the lesion guide to be displayed at a position which is easy for the doctor to see.

Returning to FIG. 3, in a case where the doctor finds a lesion for which the doctor desires to identify the boundary area, the doctor manipulates the endoscope 3 to fit the lesion within the frame of the lesion guide. Next, the doctor manipulates the endoscope 3 to give the capturing instruction. The lesion guide display unit 101 generates a still lesion image based on the capturing instruction by the doctor. The lesion guide display unit 101 outputs the lesion image and the lesion guide to the object region analysis unit 102. Also, the lesion guide display unit 101 outputs the lesion image to the boundary area visualization unit 103.

The object region analysis unit 102 estimates the lesion region in the lesion image based on the lesion image and the lesion guide. Specifically, the object region analysis unit 102 can acquire a mask image which masks the lesion region in the lesion image by using the frame and the point of the lesion guide as a prompt and inputting the frame and the point into the segmentation model together with the lesion image. The object region analysis unit 102 outputs the mask image to the boundary area visualization unit 103.

The object region analysis unit 102 can use, for example, a Segment Anything Model (SAM) published by Meta, Inc. as the segmentation model. The SAM can segment a specific region of an image by specifying a point, box, segmentation mask, or text, etc. as the prompt.

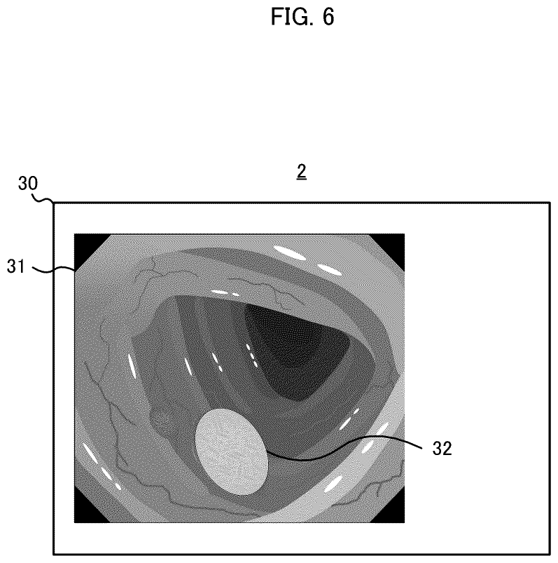

The boundary area visualization unit 103 extracts an area to be masked by the mask image from the lesion images. An image of the lesion region extracted is also referred to as a “boundary area image” hereafter. Moreover, the boundary area visualization unit 103 may generate display data as illustrated in FIG. 6 based on the lesion image and the mask image, and output the display data to the output unit 104. The output unit 104 outputs the display data to the display device 2.

### Display Example

FIG. 6 shows a display example on the display device 2. In the display example in FIG. 6, in a display area 30, a lesion image 31 and a mask area 32 are displayed. The lesion image 31 is a lesion image generated based on the capturing instruction of the doctor. The mask area 32 shows an area segmented by the object region analysis unit 102. The mask area 32 is superimposed and displayed on the lesion image 31. Such a display allows the doctor to easily identify the boundary area of the lesion.

### Endoscopy Support Process

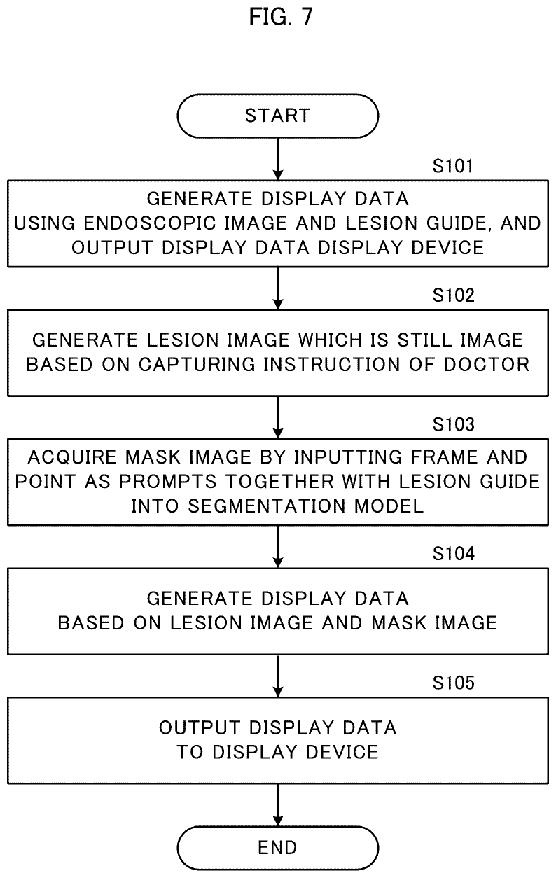

Next, an endoscopy support process for performing the above-described process will be described. FIG. 7 is a flowchart of an endoscopy support process performed by the endoscopy support device 1. This endoscopy support process is realized by the processor 11 shown in FIG. 2 which executes programs prepared in advance and operates as each element shown in FIG. 3.

The endoscopy support device 1, the endoscopic image Ic is input from the endoscope 3. The endoscopic image Ic is input to the lesion guide display unit 101. The lesion guide display unit 101 generates display data using the endoscopic image Ic and the lesion guide, and outputs the display data to the display device 2 (step S101).

In a case where the doctor finds a lesion for which the doctor desires to identify the boundary area, the doctor manipulates the endoscope 3 to fit the lesion within the frame of the lesion guide. Then, the doctor manipulates the endoscope 3 to make a capturing instruction. The lesion guide display unit 101 generates a lesion image which is a still image based on the capturing instruction of the doctor (step S102). The lesion guide display unit 101 outputs the lesion image and the lesion guide to the object region analysis unit 102. Furthermore, the lesion guide display unit 101 outputs the lesion image to the boundary area visualization unit 103.

Next, the object region analysis unit 102 acquires the mask image by inputting the frame and the point as the prompts together with the lesion guide into the segmentation model (step S103). The object region analysis unit 102 outputs the mask image to the boundary area visualization unit 103.

The boundary area visualization unit 103 generates display data based on the lesion image and the masked image, and outputs the generated display data to the output unit 104 (step S104). The output unit 104 outputs the display data to the display device 2 (step S105). After that, this endoscopy support process is terminated.

In the configuration described above, the lesion guide display unit 101 corresponds to examples of a display control means and an acquisition means, the object region analysis unit 102 corresponds to an example of the analysis means, and the boundary area visualization unit 103 and the output unit 104 correspond to examples of the boundary area visualization means and the output means.

### Modification

Next, a modification of the first example embodiment will be described. The following modifications can be combined as appropriate and applied to the first embodiment.

### Modification 1

The lesion guide display unit 101 can control a display timing of the lesion guide.

The lesion guide display unit 101 may display the lesion guide at a timing when the doctor is observing the lesion. In a case where the doctor is observing the lesion, a movement of the endoscopic image Ic, which is a video image, is reduced. Therefore, the lesion guide display unit 101 detects the movement of the endoscopic image Ic and controls whether or not to display the lesion guide. For instance, it is possible for the lesion guide display unit 101 to detect by using Optical Flow or Dlib that the movement of the endoscopic image Ic is reduced. For instance, the lesion guidance display 101 calculates Optical Flow between the frames of the endoscopic image Ic. Optical Flow indicates a moving direction and a movement amount of each point in the image. In a case where the calculated movement amount is equal to or less than a predetermined threshold value, the lesion guide display unit 101 estimates that the movement of the endoscopic image Ic is small, and displays the lesion guide.

Moreover, the lesion guide display unit 101 may display the illness guide at the timing of detecting the illness. For instance, the lesion guide analysis unit 101 can detect the lesion included in the endoscopic image Ic by using a lesion detection AI (Artificial Intelligence) previously prepared. In a case where the lesion detection AI detects the lesion, the lesion guide display unit 101 displays the lesion guide. At this time, since the lesion guide is displayed at a position set in advance, the doctor manipulates the endoscope 3 to place the lesion to identify the boundary are within the frame of the lesion guide.

### Modification 2

In the first example embodiment, the display position of the lesion guide is fixed, and the doctor manipulates the endoscope 3 to store the lesion within the frame of the lesion guide. Instead, the doctor may place the lesion within the frame of the lesion guide by moving the lesion guide itself.

If the doctor finds a lesion during the endoscopic examination, the doctor makes an instruction by voice to move the lesion guide. For instance, if the doctor gives a voice instruction “to the right”, the lesion guide display unit 101 moves the lesion guide to the right. In this case, the lesion guide display unit 101 includes a voice recognition function, by voice recognition of input voice of the doctor, and receives the voice instruction by the doctor. Also, the doctor may move the lesion guide by operating a button or a foot pedal in addition to the voice instruction. Furthermore, the lesion guide display unit 101 may move the lesion guide so as to surround the lesion detected by the lesion detection AI.

### Second Example Embodiment

Next, a second example embodiment will be described. An endoscopy system of the second example embodiment is different from that of the first example embodiment in that it is possible to identify the lesion based on the boundary area of the lesion. Note that a system configuration and a hardware configuration is the same as that of the first example embodiment, and a description thereof will be omitted.

### Functional Configuration

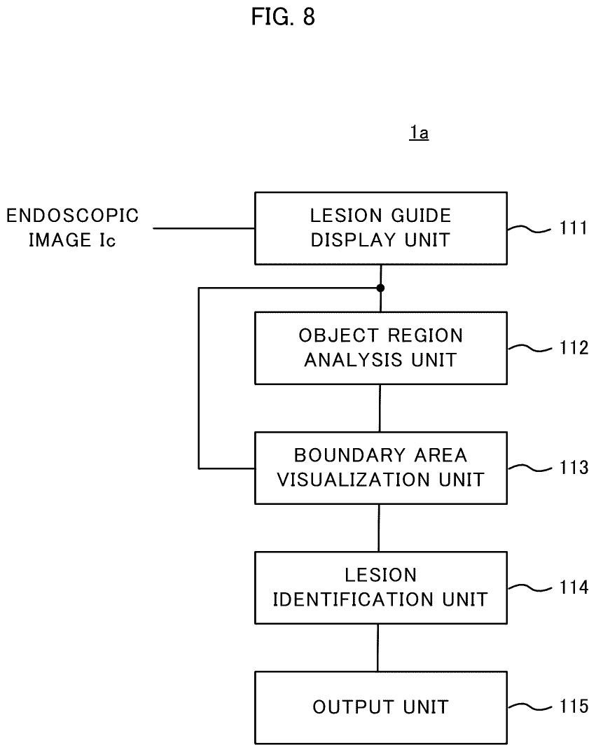

FIG. 8 is a block diagram illustrating a functional configuration of an endoscopy support device la according to the second example embodiment. The endoscopy support device la functionally includes a lesion guide display unit 111, an object region analysis unit 112, a boundary area visualization unit 113, a lesion identification unit 114, and an output unit 115.

Note that, the lesion guide display unit 111, the object region analysis unit 112, and the boundary area visualization unit 113 correspond to and perform similar operations respectively as the lesion guide display unit 101, the object region analysis unit 102, and the boundary area visualization unit 103 of the endoscopy support device, and thus, the explanations thereof will be omitted.

A boundary area image is input from the boundary area visualization unit 113 to the lesion identification unit 114. The lesion identification unit 114 identifies the boundary area image using an image recognition model prepared in advance. This image recognition model is a machine learning model trained in advance to identify an image of the lesion region as an input. Although an internal configuration of the machine learning model is arbitrary, the machine learning model may be formed by, for instance, CNN (Convolutional Neural Network).

The lesion identification unit 114 generates display data including an identification result, and outputs the display data to the output unit 115. The output unit 115 outputs the display data to the display device 2.

### Display Example

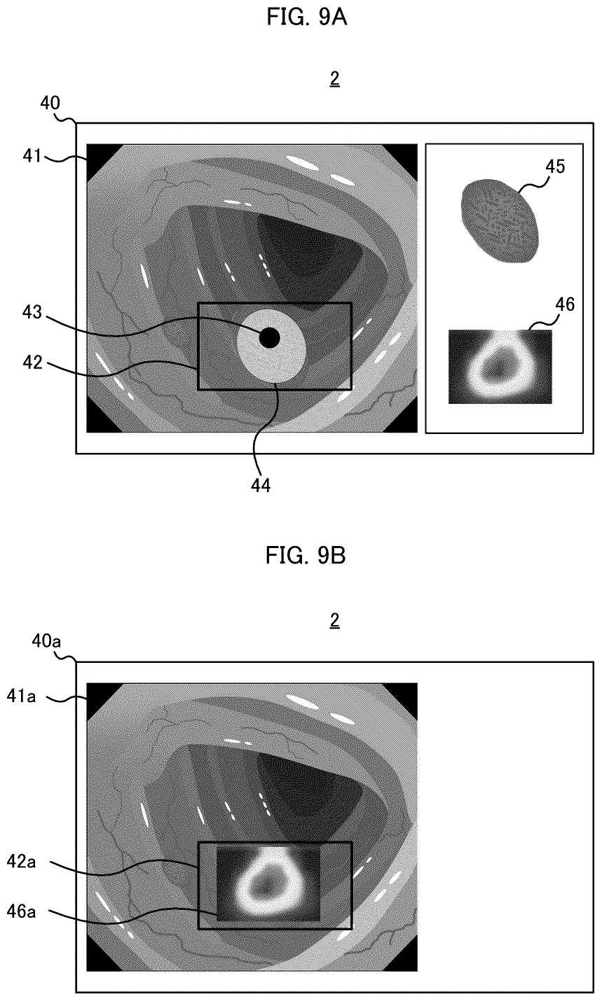

FIG. 9A and FIG. 9B illustrate display examples by the display device 2. In the display example in FIG. 9A, a lesion image 41, a lesion guide 42, an interest point 43, a mask area 44, a boundary area image 45, and an identification result 46 are displayed in the display area 40.

The lesion image 41 is a lesion image generated based on the capturing instruction of the doctor. The lesion guide 42 and the interest point 43 respectively indicate a position where the lesion guide has been displayed and a center of the lesion guide. The mask area 44 shows an area segmented by the object region analysis unit 112. The boundary area image 45 is an image of a boundary area generated by the boundary area visualization unit 113. The identification result 46 is the identification result of the boundary area image 45 by the lesion identification unit 114. In FIG. 9A, the identification result is represented by a heat map. In the heat map, a degree of cancer invasion is represented by shading or differences in color.

Moreover, FIG. 9B shows another display example by the display device 2. In the display example in FIG. 9B, the lesion image and the identification result are displayed together in one area. Specifically, in FIG. 9B, in a display area 40a, a lesion image 41a, a lesion guide 42a, and an identification result 46a are displayed. The identification result 46a is superimposed on a position of the boundary area on the lesion image 41a.

According to displays as shown in FIG. 9A and FIG. 9B, it is possible for the doctor to identify the more invasive area and then perform a biopsy. In FIG. 9A and FIG. 9B, although the identification result is shown in the heat map, the discrimination result, for instance, may be displayed in text etc.

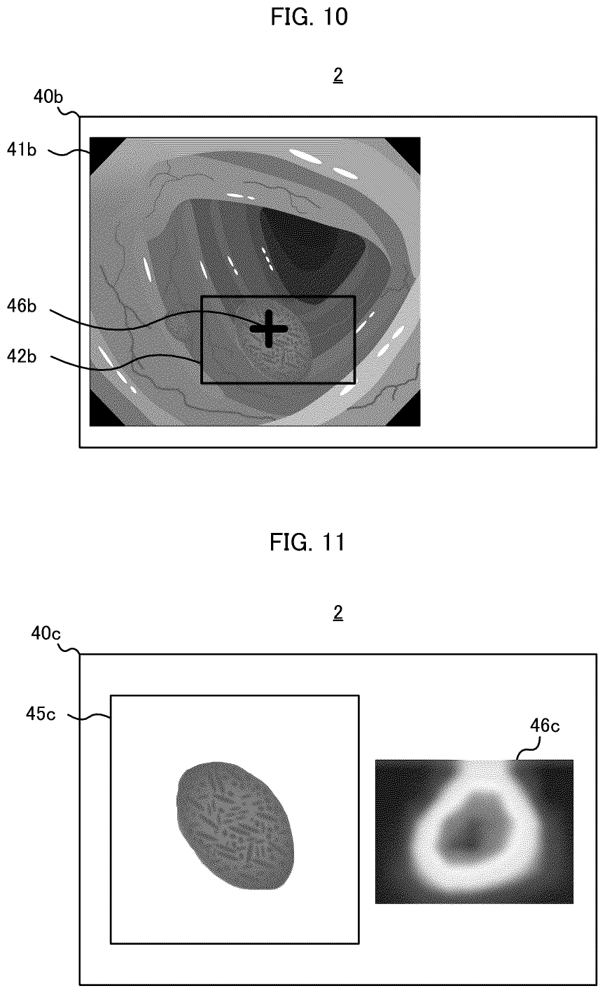

FIG. 10 shows another display example by the display device 2. This display example is also an example of a case in which the lesion image and the identification result are collectively displayed in one area. In this example, the identification result is shown by a cross. Specifically, in FIG. 10, a lesion image 41b, a lesion guidance 42b, and an identification result 46b are displayed in a display area 40b. The identification result 46b shows the most invasive part of a cancerous region. According to display shown in FIG. 10, it is possible for the doctor to identify the more invasive area and then perform the biopsy.

FIG. 11 shows another display example by the display device 2. In this example, a boundary area image 45c and an identification result 46c are displayed side by side. Specifically, in FIG. 11, the boundary area image 45c and the identification result 46c are displayed in a display area 40c. By the display as shown in FIG. 11, it is possible for the doctor to identify the boundary area of the lesion and the identification result.

In the configuration above described, the lesion identification unit 114 is an example of an identification means.

### Third Example Embodiment

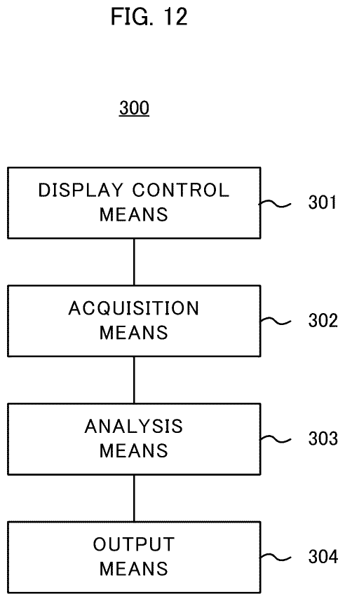

FIG. 12 is a block diagram illustrating a functional configuration of an endoscopy support device according to a third example embodiment. The endoscopy support device 300 includes a display control means 301, an acquisition means 302, an analysis means 303, and an output means 304.

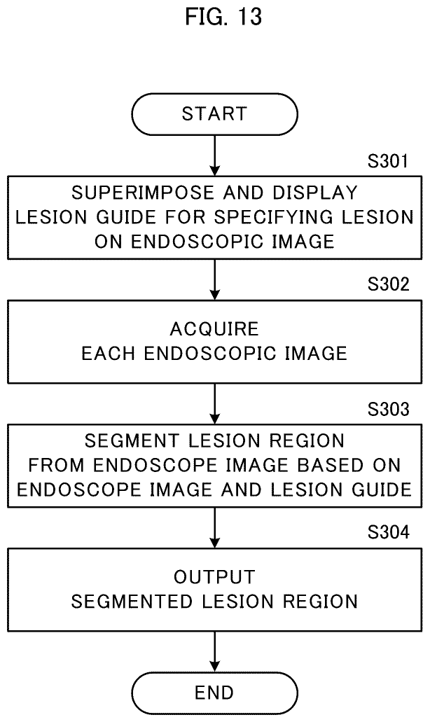

FIG. 13 is a flowchart of a process performed by the endoscopy support device according to the third example embodiment. A display control means 301 superimposes and displays a lesion guide for specifying a lesion on each endoscopic image (step S301). The acquisition means 302 acquires each endoscopic image (step S302). The analysis means 303 segments the lesion region from the endoscopic image based on the endoscopic image and the lesion guide (step S303). The output means 304 outputs the lesion region segmented (step S304).

According to the endoscopy support device 300 of the third example embodiment, it becomes possible to identify the boundary area of the lesion. Also, the endoscopy support device 300 may support decision making for a user in a medical field.

A part or all of the example embodiments described above may also be described as the following supplementary notes, but not limited thereto.

### Supplementary Note 1

An endoscopy support device comprising:
a display control means configured to superimpose and display a lesion guide for specifying a lesion in an endoscopic image;
an acquisition means configured to acquire the endoscopic image;
an analysis means configured to segment a lesion region from the endoscopic image based on the endoscopic image and the lesion guide; and
an output means configured to output the lesion region segmented.

### Supplementary Note 2

The endoscopy support device according to supplementary note 1, wherein
the lesion guide includes a frame with a predetermined size and an interest point which is a point within the frame,
the lesion is specified to be in the frame with the predetermined size, and
the analysis means generates prompts including the frame with the predetermined size and the interest point, and segments the lesion region from the endoscopic image by inputting the endoscopic image and the prompts to a segmentation model.

### Supplementary Note 3

The endoscopy support device according to supplementary note 1, wherein the analysis means acquires a mask image which masks the lesion region in the endoscopic image, as a result of segmentation.

### Supplementary Note 4

The endoscopy support device according to supplementary note 3, wherein the output means generates and outputs display data for superimposing and displaying the lesion region masked in the endoscopic image.

### Supplementary Note 5

The endoscopy support device according to supplementary note 3, further comprising:
a boundary area visualization means configured to generate a boundary area image in which the lesion region masked is extracted from the endoscopic image, and
an identification means configured to identify the boundary area image, wherein
the output means generates and outputs display data including a result of identification.

### Supplementary Note 6

The endoscopy support device according to supplementary note 3, wherein
the lesion is placed in the frame with the predetermined size based on manipulations of a user, and
the acquisition means acquires the endoscopic image based on the manipulations of the user.

### Supplementary Note 7

The endoscopy support device according to supplementary note 1, further comprising a determination means configured to determine whether or not a user is observing, based on a movement amount between endoscopic images,
wherein the display control means displays the lesion guide in a case where the user is observing.

### Supplementary Note 8

The endoscopy support device according to supplementary note 1, further comprising a lesion detection means configured to detect the lesion from the endoscopic image by using a machine learning model,
wherein the display control means displays the lesion guide in a case where a lesion detection AI detects the lesion.

### Supplementary Note 9

An endoscopy support method executed by a computer, comprising:
superimposing and displaying a lesion guide for specifying a lesion in an endoscopic image;
acquiring the endoscopic image;
segmenting a lesion region from the endoscopic image based on the endoscopic image and the lesion guide; and
outputting the lesion region segmented.

### Supplementary Note 10

A program causing a computer to execute processing of:
superimposing and displaying a lesion guide for specifying a lesion in an endoscopic image;
acquiring the endoscopic image;
segmenting a lesion region from the endoscopic image based on the endoscopic image and the lesion guide; and
outputting the lesion region segmented.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure.

This application is based upon and claims the benefit of priority from Japanese Patent Application 2024-037057, filed on Mar. 11, 2024, the disclosure of which is incorporated herein in its entirety by reference.

### DESCRIPTION OF SYMBOLS

1, 1a Endoscopy support device
2 Display device
3 Endoscope
11 Processor
12 Memory
17 Database (DB)
100 Endoscopy system
101, 111 Lesion guide display unit
102, 112 Object region analysis unit
103, 113 Boundary area visualization unit
104 Output unit
114 Lesion identification unit
115 Output unit

1. An endoscopy support device comprising:
at least one memory configured to store instructions; and
at least one processor configured to execute the instructions to:
superimpose and display a lesion guide for specifying a lesion in an endoscopic image;
acquire the endoscopic image;
segment a lesion region from the endoscopic image based on the endoscopic image and the lesion guide; and
output the lesion region segmented.

2. The endoscopy support device according to claim 1, wherein
the lesion guide includes a frame with a predetermined size and an interest point which is a point within the frame,
the lesion is specified to be in the frame with the predetermined size, and
the processor generates prompts including the frame with the predetermined size and the interest point, and segments the lesion region from the endoscopic image by inputting the endoscopic image and the prompts to a segmentation model.

3. The endoscopy support device according to claim 1, wherein the processor acquires a mask image which masks the lesion region in the endoscopic image, as a result of segmentation.

4. The endoscopy support device according to claim 3, wherein the processor generates and outputs display data for superimposing and displaying the lesion region masked in the endoscopic image.

5. The endoscopy support device according to claim 3, wherein the processor is configured to
generate a boundary area image in which the lesion region masked is extracted from the endoscopic image, and
identify the boundary area image, wherein
the processor generates and outputs display data including a result of identification.

6. The endoscopy support device according to claim 3, wherein
the lesion is placed in the frame with the predetermined size based on manipulations of a user, and
the processor acquires the endoscopic image based on the manipulations of the user.

7. The endoscopy support device according to claim 1, wherein the processor is further configured to determine whether or not a user is observing, based on a movement amount between endoscopic images,
wherein the processor displays the lesion guide in a case where the user is observing.

8. The endoscopy support device according to claim 1, wherein the processor is further configured to detect the lesion from the endoscopic image by using a machine learning model,
wherein the processor displays the lesion guide in a case where a lesion detection AI detects the lesion.

9. An endoscopy support method executed by a computer, comprising:
superimposing and displaying a lesion guide for specifying a lesion in an endoscopic image;
acquiring the endoscopic image;
segmenting a lesion region from the endoscopic image based on the endoscopic image and the lesion guide; and
outputting the lesion region segmented.

10. A non-transitory computer-readable recording medium storing a program causing a computer to execute processing of:
superimposing and displaying a lesion guide for specifying a lesion in an endoscopic image;
acquiring the endoscopic image;
segmenting a lesion region from the endoscopic image based on the endoscopic image and the lesion guide: and
outputting the lesion region segmented.
